(19) United States
(12) Patent Application Publication
(10) Pub. No.: US 2020/0116731 A1
(43) Pub. Date: Apr. 16, 2020

(54) METHOD FOR IMPROVING THE QUALITY AND QUANTITY OF OFFSPRING IN MAMMALS

(71) Applicants: Kimberly A. VONNAHME, Fargo, ND (US); Victoria C. KENNEDY, Rose Valley, PA (US)

(72) Inventors: Kimberly A. VONNAHME, Fargo, ND (US); Victoria C. KENNEDY, Rose Valley, PA (US)

(73) Assignee: NDSU Research Foundation, Fargo, ND (US)

(21) Appl. No.: 16/313,603

(22) PCT Filed: Jun. 30, 2017
(86) PCT No.: PCT/US2017/040180

§ 371 Date: Dec. 27, 2018

Related U.S. Application Data
(60) Provisional application No. 62/356,689, filed on Jun. 30, 2016.

(51) Int. Cl. G01N 33/68; A61B 5/0205; A61B 5/08; A61B 5/145

(52) U.S. Cl. CPC G01N 33/689; A61B 5/0205; A61B 2503/02; A61B 5/14542; A61B 5/0816

(57) ABSTRACT

The present disclosure relates to methods for predicting fertility and/or confirming the success of pregnancy and/or litter size in mammals. Novel methods and devices for field testing of mammal samples for pregnancy success and reproduction prosperity (fecundity) are also included.

### CROSS-REFERENCE TO RELATED APPLICATIONS

This application claims the benefit of U.S. Provisional Application Ser. No. 62/356,689, filed Jun. 30, 2016, which is incorporated by reference herein.

### BACKGROUND

In every physiological state an adequate blood volume is necessary for normal nutrient and oxygen delivery to the tissues. Similarly, blood is necessary for the collection and elimination of metabolic waste products and CO₂ from the body. In mammals an inadequate blood volume during pregnancy can have detrimental physiological consequences in the mother and the fetus.

The average blood volume (blood volume) in humans is about 71 mL/kg in men and 70 mL/kg in women (Dien, K. and C. Lentner. 1970. Documenta Geigy Scientific Tables. Ciba-Geigy, Basel, Switzerland). This means that a 170 lbs. man will have approximately 5486 mL of blood and woman of the same weight would have around 5408 mL of blood. Heavier individuals will therefore have increasing amounts of blood.

Universities and research institutions have developed values of average blood volumes of laboratory and farm/research animals. Sheep have a blood volume of around 60 mL/kg according to the NDSU IACUC (available on the world wide web at ndsu.edu/fileadm in/research/documents/IACUC/ndsu_guidelines/Policy_for_Blood_Co Ilection.pdf, (NDSU, 2013). These values vary as a consequence of species and body weight. It is specified in some of these tables that cattle up to 400 kg and horses up to 500 kg have an average blood volume of 60 mL/kg and 72 mL/kg respectively (NDSU, 2013). This specification of body weight is explained by the variation of blood volume present in different body tissues. A kg of muscle will have a higher blood volume than a kg of bone (Everett, N. B., B. Simmons, and E. P. Lasher. 1956, Distribution of blood (Fe59) and plasma (I131) volumes of rats determined by liquid nitrogen freezing. Circulation Research 4:419-424).

When the values of blood volume published by the universities and research institutions are compared with early blood volume studies similarities and differences can be found. Little difference is seen when we compare values of blood volume in dogs, 85 mL/kg according to NDSU IACUC (NDSU, 2013), and 79 mL/kg according to Courtice (Courtice, F. 1943. The blood volume of normal animals. The Journal of Physiology 102(3):290). On the other hand, blood volume in rabbits has an important difference between NDSU average values (56 mL/kg; NDSU, 2013) and the cited study (70 mL/kg; Courtice, 1943). This difference in values could be a result of blood volume measuring methods. Early studies investigating blood volume used radioactive isotopes and exsanguination to find volumes of laboratory animals (Courtice, F. 1943. The blood volume of normal animals. The Journal of Physiology 102(3):290; Goodlin, R., M. Quaife, and J. Dirksen. 1981). The significance, diagnosis, and treatment of maternal hypovolemia as associated with fetal/maternal illness. Obstetrical and Gynecological Survey 36(10):541-542). The accuracy of each method has been debated.

### Blood Volume During Pregnancy

Blood volume in rabbits increases 62% by the final period of pregnancy (Nuwayhid, B. 1979. Hemodynamic changes during pregnancy in the rabbit. American Journal of Obstetrics and Gynecology 135(5):590-596). The vast majority of studies in humans show an increase of blood volume during pregnancy (Pritchard, J. A. 1965, Changes in the blood volume during pregnancy and delivery. The Journal of the American Society of Anesthesiologists 26(4):393-399; Longo, L. 1983. Maternal blood volume and cardiac output during pregnancy: a hypothesis of endocrinologic control. American Journal of Physiology-Regulatory, Integrative and Comparative Physiology 245(5):R720-R729; Silver, H. M., M. Seebeck; and R. Carlson. 1998. Comparison of total blood volume in normal, preeclamptic, and nonproteinuric gestational hypertensive pregnancy by simultaneous measurement of red blood cell and plasma volumes. American Journal of Obstetrics and Gynecology 179(1):87-93, Torgersen, C. K. L. and C. A. Curran. 2006. A systematic approach to the physiologic adaptations of pregnancy. Critical Care Nursing Quarterly 29(1):2-19). In human studies, blood volume increase has ranged between 20% and nearly 100% (Pritchard, J. A. 1965. Changes in the blood volume during pregnancy and delivery. The Journal of the American Society of Anesthesiologists 26(4):393-399), with the increase being proportionally higher to the number of offspring carried by the mother (Pritchard, J. A. 1965. Changes in the blood volume during pregnancy and delivery. The Journal of the American Society of Anesthesiologists 26(4):393-399). Others have shown an increase between 25% and 50% (Torgersen, C. K. L. and C. A. Curran. 2006. A systematic approach to the physiologic adaptations of pregnancy. Critical Care Nursing Quarterly 29(1):2-19), The average increase in blood volume during pregnancy in humans appears to be around 45% (Pritchard, J. A. 1965. Changes in the blood volume during pregnancy and delivery. The Journal of the American Society of Anesthesiologists 26(4):393-399; Longo, L. 1983. Maternal blood volume and cardiac output during pregnancy: a hypothesis of endocrinologic control. American Journal of Physiology-Regulatory, Integrative and Comparative Physiology 245(5):R720-R729; Torgersen, C. K. L. and C. A. Curran. 2006. A systematic approach to the physiologic adaptations of pregnancy. Critical Care Nursing Quarterly 29(1):2-19).

In women, blood volume increases during pregnancy in a moderate rate in the first trimester, it increases rapidly during the second trimester with the last third experiencing a slight increase in blood volume (Pritchard, J. A. 1965. Changes in the blood volume during pregnancy and delivery. The Journal of the American Society of Anesthesiologists 26(4):393-399). The increase in hematocrit (Ht) is usually the opposite, catching up with blood volume prior to parturition (Pritchard, 1965). Plasma volume increases at high rates during the first two trimesters and stabilizes on the third, being the principal reason of blood volume increase during the first two thirds of pregnancy (Longo, L. 1983. Maternal blood volume and cardiac output during pregnancy: a hypothesis of endocrinologic control. American Journal of Physiology-Regulatory, Integrative and Comparative Physiology 245(5):R720-R729). These variations in the increase of the main components of blood during pregnancy are the explanation of the physiologically normal “pregnancy anemia” observed in women during the end of the second trimester and the beginning of the third trimester (Pritchard, J. A. 1965. Changes in the blood volume during pregnancy and delivery. The Journal of the American Society of Anesthesiologists 26(4):393-399).

In sheep, blood volume expansion during pregnancy has been debated. Some studies show blood volume expansion during pregnancy (Barcroft, J., J. Kennedy, and M. Mason. 1939. The blood volume and kindred properties in pregnant sheep. The Journal of Physiology 95(1):159-172; Caton, D., C. J. Wilcox, R. Abrams, and D. H. Barron. 1975. The circulating plasma volume of the foetal lamb as an index of its weight and rate of weight gain (g/day) in the last third of gestation. Quarterly Journal of Experimental Physiology and Cognate Medical Sciences 60(1):45-54; Daniel, S., S. James, R. Stark, and P. Tropper. 1989. Prevention of the normal expansion of maternal plasma volume: a model for chronic fetal hypoxaemia. Journal of Developmental Physiology 11(4):225-233). Others show that non-pregnant ewes and pregnant ewes exhibit small or no differences in blood volume (Metcalfe, J. and J. Parer. 1966. Cardiovascular changes during pregnancy in ewes. American Journal of Physiology—Legacy Content 210(4):821-825; Rumball, C., F. Bloomfield, and J. Harding, 2008. Cardiovascular adaptations to pregnancy in sheep and effects of periconceptional undernutrition. Placenta 29(1):89-94).

### Theoretical Mechanisms of Blood Volume Increase During Pregnancy

There are two main reasons for the importance of blood volume increase during pregnancy in females. The mother needs to compensate for the new metabolic demands of the enlarged uterus (Pritchard, J. A. 1965. Changes in the blood volume during pregnancy and delivery. The Journal of the American Society of Anesthesiologists 26(4):393-399; Torgersen, C. K. L. and C. A. Curran. 2006. A systematic approach to the physiologic adaptations of pregnancy. Critical Care Nursing Quarterly 29(1):2-19) and counteract the blood loss of parturition (Pritchard, J. A. 1965. Changes in the blood volume during pregnancy and delivery. The Journal of the American Society of Anesthesiologists 26(4):393-399; Torgersen, C. K. L. and C. A. Curran. 2006. A systematic approach to the physiologic adaptations of pregnancy. Critical Care Nursing Quarterly 29(1):2-19). An adequate blood volume increase is also necessary in order to protect mother and fetus from the deleterious effects of a reduced venous blood return and cardiac output (Pritchard, J. A. 1965. Changes in the blood volume during pregnancy and delivery. The Journal of the American Society of Anesthesiologists 26(4):393-399; Torgersen, C. K. L. and C. A. Curran. 2006, A systematic approach to the physiologic adaptations of pregnancy. Critical Care Nursing Quarterly 29(1):2-19). Pregnant women can handle more blood loss that non-pregnant women. They can lose up to 35% of their blood volume before showing signs of hypovolemia (Pritchard, J. A. 1965. Changes in the blood volume during pregnancy and delivery. The Journal of the American Society of Anesthesiologists 26(4):393-399; Torgersen, C. K. L. and C. A. Curran. 2006. A systematic approach to the physiologic adaptations of pregnancy. Critical Care Nursing Quarterly 29(1):2-19).

While it is well established why maternal blood volume increase would need to occur, there is still debate on how blood volume increases. There are currently two theories that attempt to explain blood volume expansion: the decreased vascular resistance theory and the endocrine theory.

The decreased vascular resistance theory describes a mechanism by which blood volume could increase during pregnancy (Schrier, R. W. and V. A. Briner. 1991. Peripheral arterial vasodilation hypothesis of sodium and water retention in pregnancy: implications for pathogenesis of preeclampsia-eclampsia. Obstetrics and Gynecology 77(4):632-639; Duvekot, J. J., E. C. Cheriex, F. A. Pieters, P. P. Menheere, H. J. Schouten, and L. L. Peeters. 1995. Maternal volume homeostasis in early pregnancy in relation to fetal growth restriction. Obstetrics and Gynecology 85(4361-367) When the female becomes pregnant a new vascular system is added to the main vascular system (Schrier, R. W. and V. A. Briner. 1991. Peripheral arterial vasodilation hypothesis of sodium and water retention in pregnancy: implications for pathogenesis of preeclampsia-eclampsia. Obstetrics and Gynecology 77(4):632-639; Duvekot, J. J., E. C. Cheriex, F. A. Pieters, P. P. Menheere, H. J. Schouten, and L. L. Peeters. 1995. Maternal volume homeostasis in early pregnancy in relation to fetal growth restriction. Obstetrics and Gynecology 85(3):361-367). This new addition decreases the total vascular resistance of the cardiovascular system of the mother (Schrier, R. W. and V. A. Briner. 1991. Peripheral arterial vasodilation hypothesis of sodium and water retention in pregnancy: implications for pathogenesis of preeclampsia-eclampsia. Obstetrics and Gynecology 77(4):632-639; Duvekot, J. J., E. C. Cheriex, F. A. Pieters, P. P. Menheere, H. J. Schouten, and L. L. Peeters. 1995. Maternal volume homeostasis in early pregnancy in relation to fetal growth restriction. Obstetrics and Gynecology 85(4361-367). This in turn increases the heart rate in the mother, which activates the plasma volume regulating mechanisms in the liver, kidneys, and adrenal glands (Schrier, R. W and V. A. Briner. 1991, Peripheral arterial vasodilation hypothesis of sodium and water retention in pregnancy: implications for pathogenesis of preeclampsia-eclampsia. Obstetrics and Gynecology 77(4):632-639; Duvekot, J. J., E. C. Cheriex, F. A. Pieters, P. P. Menheere, H. J. Schouten, and L. L. Peeters. 1995. Maternal volume homeostasis in early pregnancy in relation to fetal growth restriction. Obstetrics and Gynecology 85(4361-367). As plasma volume increases, blood volume increases as well (Schrier, R. W. and V. A. Briner. 1991. Peripheral arterial vasodilation hypothesis of sodium and water retention in pregnancy: implications for pathogenesis of preeclampsia-eclampsia. Obstetrics and Gynecology 77(4):632-639; Duvekot, J. J., E. C. Cheriex, F. A. Pieters, P. P. Menheere, H. J. Schouten, and L. L. Peeters. 1995. Maternal volume homeostasis in early pregnancy in relation to fetal growth restriction. Obstetrics and Gynecology 85(3):361-367).

The endocrine control theory (Longo, L. 1983. Maternal blood volume and cardiac output during pregnancy: a hypothesis of endocrinologic control, American Journal of Physiology-Regulatory, Integrative and Comparative Physiology 245(5):R720-R729) suggests a fetal influence on blood volume in the pregnant female. As gestation advances, the fetus, and its adrenal glands increase in size (Longo, L. 1983. Maternal blood volume and cardiac output during pregnancy: a hypothesis of endocrinologic control. American Journal of Physiology-Regulatory, Integrative and Comparative Physiology 245(5):R720-R729′ As adrenal gland size increases there is an increasing production of dehydroepiandrosterone, a hormone that stimulates estradiol production in the mother (Longo, L. 1983. Maternal blood volume and cardiac output during pregnancy: a hypothesis of endocrinologic control. American Journal of Physiology-Regulatory, Integrative and Comparative Physiology 245(5):R720-R729). Estradiol then stimulates the renin-angiotensin system, which ultimately increases plasma volume (Longo, L. 1983. Maternal blood volume and cardiac output during pregnancy: a hypothesis of endocrinologic control. American Journal of Physiology-Regulatory, Integrative and Comparative Physiology 245(5):R720-R729). This theory also suggests a mechanism through which erythrocytes increase during pregnancy. During gestation, placental size increases and as placental tissue grows there is an increasing production of somatomammotropin (i.e. placental lactogen) and progesterone (Longo, L. 1983. Maternal blood volume and cardiac output during pregnancy: a hypothesis of endocrinologic control. American Journal of Physiology-Regulatory, Integrative and Comparative Physiology 245(5):R720-R729′ These two hormones stimulate the production of erythropoietin in the mother, which finally stimulates the production of erythrocytes (Longo, L. 1983. Maternal blood volume and cardiac output during pregnancy: a hypothesis of endocrinologic control. American Journal of Physiology-Regulatory, Integrative and Comparative Physiology 245(5):R720-R729).

### Consequences of an Inadequate Blood Volume Increase During Pregnancy

In women, failure to increase blood volume during pregnancy has been related to pregnancy-induced toxemia (preeclampsia), fetal growth retardation, and premature labor (Goodlin, R., M. Quaife, and J. Dirksen. 1981. The significance, diagnosis, and treatment of maternal hypovolemia as associated with fetal/maternal illness. Obstetrical and Gynecological Survey 36(10):541-542). Similarly, risks of blood loss during parturition are greater with women losing up to 1 L of blood during normal labor and 1.5 L or more during a cesarean section (Pritchard, J. A. 1965. Changes in the blood volume during pregnancy and delivery. The Journal of the American Society of Anesthesiologists 26(4):393-399). Failure to increase blood volume during pregnancy could be the cause or the consequence of many feta-maternal illnesses. An inadequate function of the mechanisms necessary to increase blood volume in a state of decreased vascular resistance could consequently increase heart rate and produce vasoconstriction (Lund, C. J. and J. C. Donovan. 1967. Blood volume during pregnancy. American Journal of Obstetrics and Gynecology 98(3):393-403; Goodlin, R., M. Quaife, and J. Dirksen. 1981. The significance, diagnosis, and treatment of maternal hypovolemia as associated with fetal/maternal illness. Obstetrical and Gynecological Survey 36(10):541-542′ This could increase blood pressure and therefore be one of the causes of preeclampsia (Lund, C. J, and J. C. Donovan. 1967. Blood volume during pregnancy. American Journal of Obstetrics and Gynecology 98(3):393-403; Goodlin, R., M. Quaife, and J. Dirksen. 1981. The significance, diagnosis, and treatment of maternal hypovolemia as associated with fetal/maternal illness. Obstetrical and Gynecological Survey 36(10):541-542). Another way of understanding an inadequate blood volume increase could be by the existence of a reduced vasodilatory capacity of the cardiovascular system of the mother previous to pregnancy (Assali, N. and D. Vaughn. 1977. Blood volume in pre-eclampsia: fantasy and reality. American Journal of Obstetrics and Gynecology 129(4):355-359; Campbell, D. M. and A. J. Campbell. 1983. Evans Blue disappearance rate in normal and pre-eclamptic pregnancy. Clinical and Experimental Hypertension. Part B; Hypertension in Pregnancy 2(1):163-169). This would prevent blood volume increase and favor preeclampsia due to a reduced vessel compliance (Assali, N, and D. Vaughn. 1977. Blood volume in pre-eclampsia: fantasy and reality. American Journal of Obstetrics and Gynecology 129(4):355-359; Campbell, D. M. and A. J. Campbell. 1983, Evans Blue disappearance rate in normal and pre-eclamptic pregnancy, Clinical and Experimental Hypertension. Part B: Hypertension in Pregnancy 2(1):163-169). Other feto-maternal illnesses such as fetal growth retardation could be a consequence of this state (Assali, N. and D. Vaughn. 1977. Blood volume in pre-eclampsia: fantasy and reality. American Journal of Obstetrics and Gynecology 129(4):355-359; Campbell, D. M. and A. J. Campbell. 1983. Evans Blue disappearance rate in normal and pre-eclamptic pregnancy. Clinical and Experimental Hypertension. Part B: Hypertension in Pregnancy 2(1):163-169).

In accordance with the idea of inadequate blood volume increase as the cause of pregnancy related illnesses, some studies have shown that fetal growth retardation can happen independent of preeclamptic states but with failure to increase blood volume (Lund, C. J. and J. C. Donovan. 1967. Blood volume during pregnancy. American Journal of Obstetrics and Gynecology 98(3):393-403; Grunberger et al., 1979. Maternal Hypertension, Fetal Outcome in Treated and Untreated Cases. Gynecol Obstet Invest 10:32-38). Similarly, pregnant women with hypovolemia can have all ranges of blood pressure with hypertension probably expressing cardiac compensation and hypotension representing malnutrition (Lund, C. J, and J. C. Donovan. 1967. Blood volume during pregnancy. American Journal of Obstetrics and Gynecology 98(3):393-403).

A sheep study done in 2008 showed that periconceptional undernutrition does not affect blood volume on days 65 and 120 of gestation (Rumba C., F. Bloomfield, and J. Harding. 2008. Cardiovascular adaptations to pregnancy in sheep and effects of periconceptional undernutrition. Placenta 29(1):89-94). However this study did not measure blood volume during the period of nutrient restriction and did not measure blood volume in adequately fed pregnant ewes.

As mentioned before, when females become pregnant a new vascular system is added to the main maternal vascular system (Schrier, R. W. and V. A. Briner. 1991. Peripheral arterial vasodilation hypothesis of sodium and water retention in pregnancy: implications for pathogenesis of preeclampsia-eclampsia. Obstetrics and Gynecology 77(4):632-639; Duvekot, J. J., E. C. Cheriex, F. A. Pieters, P. P. Menheere, H. J. Schouten, and L. L. Peeters. 1995. Maternal volume homeostasis in early pregnancy in relation to fetal growth restriction, Obstetrics and Gynecology 85(3):361-367). This new vascular system is comprised of the fetal and placental vessels that have specific anatomical and physiological characteristics.

### SUMMARY

It is unknown whether plasma volume expansion occurs and whether this plasma volume expansion could be used as a tool to determine successful attainment of pregnancy (fertility), number of fetuses in the uterus (litter size), and/or successful continuation of pregnancy throughout the normal gestation length of a female. What is particularly novel about our findings is that it appears that hematocrit (or packed red cell volume) near to, or at the time of breeding (or artificial insemination) can predict future success of pregnancy (fertility) and/or litter size.

There is no evidence that pregnancy success in animals can be predicted by measuring hematocrit levels immediately prior to insemination. Also, there is no evidence of predicting litter size by measuring hematocrit levels at the time of insemination or immediately after insemination.

The current method for detection of pregnancy in animals is the use of ultrasonography, and this is performed with accuracy well into the pregnancy (e.g., at least after ⅓ of the pregnancy length has passed). The present disclosure shows that measuring hematocrit levels immediately or shortly before and/or after insemination can provide predictive data on the success and fecundity of livestock. Moreover, the potential for the use of pulse oximetry can serve to determine pregnancy earlier in pregnancy, and with reduced invasiveness or restraint.

The present disclosure relates to methods for predicting the success of pregnancy (fertility), attainment of pregnancy, and/or litter size in mammals. Novel methods and devices for field testing of mammal samples for pregnancy success and reproduction prosperity (fecundity) are also included.

One aspect of the disclosure is a method for predicting the success of pregnancy in a mammal comprising a) obtaining a relevant sample near the time of insemination, b) determining a physiological measurement from the sample and c) comparing the physiological measurement to the same physiological measurement of a control mammal. In one embodiment, the control mammal is the same animal being evaluated for pregnancy, but the relevant sample is taken before insemination. In another embodiment, the control mammal is the same species as the mammal being evaluated for pregnancy, but the physiological measurement is one that would be expected for a mammal that will not or has not become pregnant after insemination.

The range of hematocrit values may be specific to the species that is being investigated. That being said, cattle, sheep, and swine will have values that fall between 20 and 90% hematocrit at various times during their life cycle. It has been determined (as demonstrated in the Examples herein), that hematocrit values are 10 to 50% decreased in animals that are pregnant, are predicted to be pregnant or have increased litter size compared to non-pregnant animals, animals that do not acheive pregnancy, or have reduced litter size.

Another aspect of the disclosure is a method for confirming the success of pregnancy in a mammal comprising a) obtaining a relevant sample after the time of insemination, b) determining a physiological measurement from the sample and c) comparing the physiological measurement to the same physiological measurement of a control mammal. In one embodiment, the control mammal is the same animal being evaluated for pregnancy, but the relevant sample is taken before insemination. In another embodiment, the control mammal is the same species as the mammal being evaluated for pregnancy, but the physiological measurement is one that would be expected for a mammal that has not become pregnant after insemination.

Another aspect of the disclosure is a method for predicting litter size in a mammal comprising a) obtaining a relevant sample near the time of insemination, b) determining a physiological measurement from the sample and c) comparing the physiological measurement to those that would be expected for a mammal that would have a litter of a known size.

Another aspect of the disclosure is a method for identifying qualified candidates for successful embryo transplants from an embryo donor comprising the above method.

Another aspect of the disclosure is a kit for predicting a successful pregnancy (fertility) in a mammal comprising a) instructions for collecting relevant samples, b) instructions for obtaining physiological measurements and c) instructions for interpreting the physiological measurements and or bodily parameters to predict a successful pregnancy. Optionally, implements can be included with the kit.

Another aspect of the disclosure is a kit for confirming a successful pregnancy in a mammal comprising a) instructions for collecting relevant samples, b) instructions for obtaining physiological measurements and c) instructions for interpreting the physiological measurements and or bodily parameters to confirm a successful pregnancy. Optionally, implements can be included with the kit.

Another aspect of the disclosure is a kit for predicting litter size in a mammal comprising a) instructions for collecting relevant samples, b) instructions for obtaining physiological measurements and c) instructions for interpreting the physiological measurements and or bodily parameters to predict a litter size in a mammal. Optionally, implements can be included with the kit.

Another aspect of the disclosure is a biochemical device comprising a compact sensor that can analyze a small amount of a relevant sample and determine a physiological measurementhe sample, particularly when the relevant sample is a physiological sample.

Another aspect of the disclosure is a biometric device comprising a compact sensor that can determine a physiological measurement in a relevant sample, especially when the relevant sample is access to a bodily parameter.

Optionally, the compact sensor is in communication with a processing device to generate the physiological measurement and/or analyze a physiological measurement against standards to predict the pregnancy of a mammal and/or size of a litter.

Additional optional features of the present disclosure and preferred embodiments are described in more detail below and in the examples and claims also enumerated below.

### DESCRIPTION OF THE FIGURES

FIG. 1 shows hematocrit levels in pregnant ewes carrying singletons lambs (black dots), ewes carrying twin lambs (white dots), or non-pregnant (never bred), cycling ewes (red square). Means±standard error of the mean are presented. Data were analyzed using SAS 9.2. There was a significant effect of fetal number (P=0.03).

FIG. 2 shows hematocrit (Ht) values prebreeding (2 days prior to breeding) and resulting litter size at birth. Litter size is presented as fully formed piglets (Live+still born; top panel) and just live born piglets (bottom panel). Each dot represents a female on study. Data were analyzed using SAS 9.2. The correlation and P values for each analysis is presented in the bottom left hand side of the graphs.

FIG. 3 shows hematocrit values obtained on the day of breeding in dairy cows and heifers (n=120). Breeding groups: 1, females returned to heat in 21 days; 2, females conceived, but lost pregnancy by day 30 after breeding; 3, females conceived but lost pregnancy by 60 days of gestation; 4, females conceived and successfully carried their calves to term or past 60 days of gestation (some have not calved yet). ^{ab}Means±SEM with different superscripts differ; P<0.05. Data were analyzed using SAS 9.2.

### DETAILED DESCRIPTION OF ILLUSTRATIVE EMBODIMENTS

The present disclosure relates to methods for predicting the success of pregnancy and/or litter size of a mammal. Novel methods and devices for field testing of mammal samples for predicting pregnancy (fertility), pregnancy success (attainment of pregnancy) and reproduction prosperity (fecundity) are also included.

A first aspect of the disclosure is methods for predicting the success of pregnancy in a mammal comprising obtaining a relevant sample near the time of insemination, determining a physiological measurement from the sample and comparing the physiological measurement to those that would be expected for a mammal that will not or has not become pregnant after insemination.

A relevant sample could be any physiological sample or access to a bodily parameter. For example, a physiological sample can be a bodily fluid, including but not limited to, blood, or urine, or feces, preferably a sample containing blood or blood components. From the physiological sample, a physiological measurement can be obtained such as hematocrit levels, hemoglobin, break down products of hemoglobin and bilirubin (e.g., stercobilin, urobilin, porphyrins). A bodily parameter includes a non-chemical, and usually non-invasive, measurement of a physiological function, such as heart rate, respiration rate, blood pressure, or oxygen saturation of the blood. Preferred relevant samples include blood and oxygen saturation. Oxygen saturation can therefore be determined from a physiological sample or a bodily parameter and can be determined chemically from a blood sample or measuring a bodily parameter, e.g., using an oximeter. Preferred physiological measurements include the measurement of hematocrit levels and/or oxygen saturation levels to predict the likelihood of a successful pregnancy of an inseminated mammal. A suitable amount of a relevant sample is either a sufficient physical amount of a physiological sample or a sufficient amount of time in accessing a bodily parameter for a physiological measurement to be determined. It will be an amount that is sufficient for the relevant sample and the physiological measurement being determined as is known to those of skill in the art.

Physiological measurements can be made using chemical methods and/or instrumentation that is known to those of skill in the art. In particular, measuring hematocrit levels and/or oxygen saturation are well established and further exemplified in the Examples below.

Predicting the success of pregnancy means determining the likelihood of a successful pregnancy based upon physiological measurements near the time of insemination, preferably prior to or within a short time after a mammal has been inseminated, known as the prediction window. The physiological measurements are taken from a female mammal.

The prediction window is typically from 30 days prior to insemination to 60 days after insemination, preferably from 7 days prior to insemination to 30 days after insemination, more preferably from 2 days prior to insemination to 21 days after insemination, and most preferably from 0 days prior to insemination to14 days after insemination. Insemination can be by natural or artificial insemination.

This first aspect of the disclosure can also be used to confirm pregnancy in a female mammal. This method includes a method for confirming the success of pregnancy in a mammal comprising a) obtaining a relevant sample after the time of insemination, b) determining a physiological measurement from the sample and c) comparing the physiological measurement to the same physiological measurement that would be expected for a mammal that has not become pregnant after insemination. This is determined by measuring hematocrit, pulse oximetry, or a combination thereof, and confirming pregnancy with such methods as ultrasonography, progesterone analysis, and birth.

The mammal can be any mammal, including a human, an animal used in agriculture (e.g., livestock), a domesticated mammal, or an undomesticated animal (e.g., wildlife). Mammal refers to a warm-blooded vertebrate that has hair, and lactates. The disclosure is particularly suited for maximizing the number of progeny of livestock and domesticated animals in the most productive way to minimize the time a mammal is not gestating any offspring to maximize the size and value of the livestock. This leads to maximizing the number of livestock or domesticated animals, preferably livestock, in a breeding population such as, for example, a herd, flock, pride, pack or band. Preferred livestock include bovine species (both dairy and meat cattle), bison, sheep, goats, pigs, horses, llama, alpaca, rabbits, and mink, preferably dairy cattle, meat cattle, sheep and pigs. Preferred domesticated animals include most companion animals, preferably dogs and cats.

The disclosure can also be used to assess the breeding population of wildlife in the wild and take corrective steps to increase or decrease the size of the breeding population in the wild. Increasing the size of a wildlife breeding population could be desirable for threatened or endangered species or wildlife species located in non-natural habitats such as zoos. In some cases, this disclosure could potentially be helpful in discriminating between true pregnancy and pseudopregnancy, which is a huge problem in many canines, big cat species and hibernating bears. In other species where there has been more success with reproductive technologies, zoos still must often wait for the majority of pregnancy before confirming its success. Decreasing the size of a breeding wildlife population could be desirable for invasive or pest species. While methods of birth control and decreasing pregnancies in many of these species are already in use, confirmation of pregnancy prevention is still needed. Wildlife species can be any mammal that is not livestock or a domesticated animal, preferably threatened species, endangered species, pest species, invasive species, game and zoo animals, preferably threatened species, endangered species and zoo animals.

The disclosure can also be used to assess reproductive capacity in women. This could be particularly useful in artificial reproductive technologies such as super ovulation for collection of oocytes to perform in vitro fertilization and embryo transfer. Moreover, the disclosure could be used as a predictor of pregnancy success, perhaps at a much earlier stage of pregnancy, or even successfully determining a fertile ovulation.

Successful pregnancies are predicted if a physiological measurement is lower than what would be expected in a mammal that will not or has not become pregnant. Successful pregnancies are predicted if the oxygen saturation levels are lower than what would be expected in a mammal that will not or has not become pregnant. Successful pregnancies can be predicted by a decrease in hematocrit values compared to non-pregnant animals of at least 15%, at least 20%, at least 25%, or at least 30%. In one embodiment, successful pregnancies can be predicted by a decrease in hematocrit values compared to non-pregnant animals of no greater than 50%, no greater than 45%, no greater than 40%, or no greater than 35%. Successful pregnancies can be predicted by a decrease in pulse oximetry measurements in pregnant compared to non-pregnant animals of at least 10%, at least 15%, or at least 20%. In one embodiment, successful pregnancies can be predicted by a decrease in pulse oximetry measurements compared to non-pregnant animals of no greater than 40%, no greater than 35%, or no greater than 30%. However, any value of reduction that is at least as great as the standard deviation or error bars for a set of non-pregnant mammals would be considered a significant reduction to predict pregnancy.

A second aspect of the disclosure is methods for predicting the litter size in a mammal comprising obtaining a relevant sample near the time of insemination, determining a physiological measurement from the sample and comparing the physiological measurement to those that would be expected for a mammal that would have a litter of known size.

This second aspect of the disclosure is related to the first aspect of the disclosure with the following variations. First, the prediction window to predict litter size is typically from 10 days prior to insemination to 60 days after insemination, preferably from 7 days prior to insemination to 45 days after insemination, more preferably from 2 days prior to insemination to 30 days after insemination, and most preferably from 0 days prior to insemination to 21 days after insemination. Insemination can be by natural or artificial insemination.

Second, litter size can be predicted by a decrease in hematocrit levels and/or oxygen saturation and the correlation to predicting litter size is a 15 to 50% decrease in hematocrit values compared to non-pregnant animals, or a 10 to 40% decrease in pulse oximetry measurements in pregnant compared to non-pregnant animals. However, any value of reduction that is at least as great as the standard deviation or error bars for a set of non-pregnant mammals would be considered to be a significant reduction to predict litter size. Alternatively, the litter size of a mammal can be predicted by comparing the hematocrit levels and/or oxygen saturation levels to mammal having a litter of known size. To determine what would be expected for a mammal having different sizes of litters, physiological measurements can be taken of pregnant mammals during the prediction window and the value of a physiological measurement, such as hematocrit or oxygen saturation level, can be correlated with the resulting litter size at birth. For instance, for ewes, the hematocrit and/or oxygen saturation level can be determined for pregnant ewes that eventually have a litter size of 1, 2, or 3. For pigs, the hematocrit and/or oxygen saturation level can be determined for pregnant sows that eventually have a litter size of 6 to 16 fully formed piglets (or 4 to 15 live born piglets)

A third aspect of the present disclosure is a method of identifying qualified candidates for successful embryo transfers from an embryo donor. This is especially useful when breeding valuable mammals or a mammal genotype limited in a population. For example, high yielding dairy breeds, lean cattle, etc. are an example of a valuable mammal. Genetically engineered mammals would be an example of a genotype that is limited in the population. The use of some animals to produce biological molecules in milk and blood for human therapeutic uses has been accomplished. Also, some mouse models of human disease can be hard to breed in large numbers and this aspect of the disclosure would facilitate production of larger numbers of such mammals. Currently, there are no known markers for an ideal donor female. It is our hypothesis that a female with greater blood volume would equate to a better recipient of the donor embryo.

Using the first aspect of the present disclosure, one could predict those embryo recipients (surrogates) that have an enhanced opportunity for achieving a successful pregnancy and/or an increased litter size.

A fourth aspect of the disclosure are kits for performing the methods of the disclosure. Such kits typically comprise instructions and optional implements for collecting relevant samples, instructions for obtaining physiological measurements and instructions for interpreting the physiological measurements and or bodily parameters to predict fertility, a successful pregnancy, and/or litter size. Instructions for collecting relevant samples can include the type of physiological sample to collect or the bodily parameter to access, the amount or type of sample to be collected and proper storage conditions for any physiological sample. If the relevant sample is accessible to a bodily parameter, the instructions can include how to access the bodily parameter and how the bodily parameter is to be measured, instructions for interpreting the physiological measurements and/or bodily parameters can include quantitative or relative variations in the physiological measurement or bodily parameter that indicates fertility, a successful pregnancy, and/or litter size. Such kits can optionally also include the devices of the fourth aspect of the disclosure and instructions for their use.

A fifth aspect of the disclosure are novel devices to be used in performing the methods of the disclosure. Although many of the techniques for determining the physiological measurements of method aspects of the disclosure are known to those of skill in the art, the present disclosure also includes novel devices for conveniently determining those physiological measurements, particularly remotely in the field or at the location of the of the relevant sample. One such device is a biochemical device comprising a compact sensor that can analyze a small amount of a relevant sample and determine a physiological measurement in the sample, particularly when the relevant sample is a physiological sample. A second device is a biometric device comprising a compact sensor that can determine a physiological measurement in a relevant sample, especially when the relevant sample is access to a bodily parameter.

The biochemical device of the present disclosure comprises a compact sensor that can perform a chemical and/or spectrophotometric analysis of a relevant sample, preferably a physiological sample. A compact sensor can perform an analysis of a sample to determine a physiological measurement as is known in the art. The compact sensor is novel in that it is small enough to be used in the field, is mobile, is robust to be used in the field and can generate a physiological measurement from a sample. Optionally, the compact sensor can be in communication with a processing device to generate the physiological measurement and/or analyze a physiological measurement against standards to predict the pregnancy of a mammal and/or size of a litter. The processing device may be used simultaneously with, or subsequent to, the determination of the physiological measurement. The processing device is preferably a tablet-like device or a mobile telecommunications device (a smart phone). Upon receiving the physiological measurement from the compact sensor, the processing device can compare the value of the physiological device to physiological measurements that would be expected for a mammal that will not or has not become pregnant, or depending on the magnitude of variation from the expected value for a mammal that will not or has not become pregnant, or can predict the litter size.

The biometric device of the present disclosure is similar to the biochemical device aspect of the present disclosure with the following modifications. First, the compact sensor determines a physiological measurement by access to a bodily parameter instead of a physiological sample. Second, the configuration of the biometric device is such that it can be used to obtain the physiological measurement in a first step, then attached to the processing device to analyze a physiological measurement against standards to predict the pregnancy of a mammal and/or size of a litter. A preferred embodiment of the biometric device comprises a hand or hand appendage conforming material with the compact sensor attached to or embedded in a convenient location for easily accessing a location on a mammal so that a bodily parameter can be accessed to obtain a physiological measurement. A more preferred embodiment of the biometric device would be glove with a compact sensor located near the tip of the index finger for accessing a bodily parameter. A preferred bodily parameter would be oxygen saturation using a pulse oximetry compact sensor. Preferred locations on a mammal to access with the biometric device include an udder or vulva, preferably the vulva. Accessing the vulva to measure oxygen saturation would be a particularly preferred embodiment.

For such a glove embodiment, it would be important that the hand conforming material insulates the compact sensor from the index finger of the person accessing the bodily parameter to assure that any measurement of oxygen saturation is a measurement of the mammal and not the person accessing the bodily parameter. Another preferred embodiment would be locating the compact sensor of the biometric device on a probe, such as a milking device, a pole, or a robotic device, especially for obtaining access to a bodily parameter of, for example, a dairy cow or wildlife.

Communication of the compact sensor and the processing device can be hard wired during the obtainment of data (for example in the case of a milking device) or it can be uploaded later. Communication can also be by wireless communication, including blue tooth technology. Uploading the data collected to a processing device containing a database of values collected for a particular species will allow a continual updating of significance for the difference in a measurement of a physiological function or a bodily parameter is different enough (significant) than what is expected in a mammal that will not or has not become pregnant.

The methods and devices of the present disclosure are useful for predicting the reproduction quality and quantity of a mammal population. It can also be used to improve or inhibit the size of a breeding population of mammals. It can also improve the efficiency of increasing the size of a breeding population of breeding mammals in less time.

The details of one or more embodiments of the presently-disclosed subject matter are set forth in this document, Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

When the term “including” or ‘including, but not limited to” is used, there may be other non-enumerated members of a list that would be suitable for the making, using or sale of any embodiment of this disclosure.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the disclosure(s) belong.

All patents, patent applications, published applications and publications, GenBank sequences, databases, websites and other published materials referred to throughout the entire disclosure herein, unless noted otherwise, are incorporated by reference in their entirety.

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present disclosure as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a” “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

### EXAMPLES

### Example 1: Measuring Hematocrit Levels in a Mammal Blood Sample Early in Pregnancy to Confirm Pregnancy and Enumerate Fetal Number

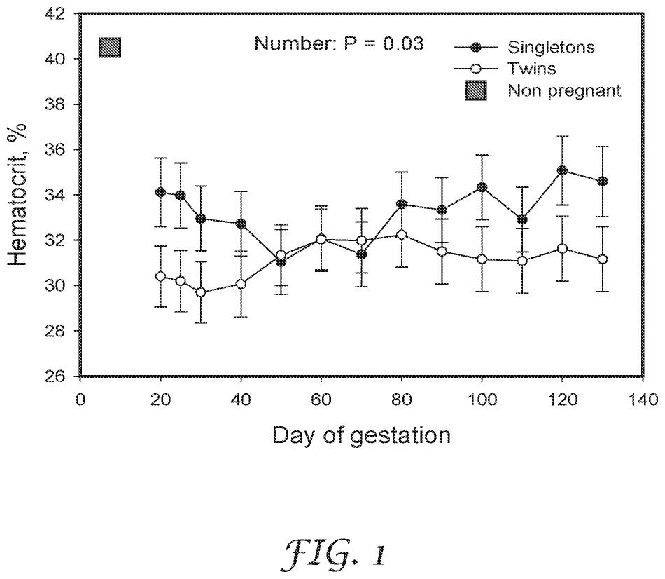

The original hypothesis is that we would detect differences in hematocrit levels during late gestation in ewes carrying singletons and twins as it has been reported that there is an increase in plasma volume expansion in late pregnant females (including ewes, rabbits, mice and humans) compared to non-pregnant females. The experiment was designed to begin hematocrit testing when ultrasonography for detection of pregnancy, and enumeration of fetuses began, which was on day 20 after breeding (gestation length is 150 days). The expected outcome was the ewes carrying twins would have decreased hematocrit during late gestation compared to ewes carrying singletons because of plasma volume expansion. Blood samples (˜10 mL in an EDTA vacutainer tube) were collected from the jugular vein on days 20, 25, 30, 40, 50, 60, 70, 80, 90, 100, 110, 120, and 130 of gestation. As early as day 20, we were able to detect differences between ewes that were carrying singletons and twin lambs. Our hypothesis that ewes carrying more lambs would have decreased hematocrit was correct. However, we were surprised to see that occurred as early as day 20. In order to determine if our values were different than non-pregnant (ewes that were used were never bred and their estrous cycle was being monitored; estrous cycle length in the ewe is ˜17 days), we collected blood samples (from the jugular vein) on day 5 and day 10 of the estrous cycle. Hematocrit was determined using microhematocrit capillary tubes and a centrifuge, Length of red blood cells and total sample were measured with a digital calipers. The length of the RBCs were divided by the total sample volume and multiplied by 100 to obtain hematocrit %. As FIG. 1 indicates, there was a significant increase of hematocrit on these days compared to day 20 samples of pregnancy, regardless of fetal number.

This led to a new hypothesis: that hematocrit levels would decrease due to plasma volume expansion which would occur during the time of maternal recognition. Our goal was to determine in sheep when hematocrit levels decreased compared to non-pregnant controls and how early in pregnancy we can enumerate. Currently, data that has already been collected suggests that we can determine by day 20 after breeding.

To predict litter size in a mammal, blood samples are taken prior to insemination of the animal (usually just prior to insemination on the same day of insemination) and hematocrit levels are determined.

In order to determine the earliest we can predict litter size in the ewe, hematocrit measurements (as described above) are taken just prior to the time of breeding and through the first 3 week post breeding. Moreover, we gather pulse oximetry measurements each time we test hematocrit to determine the effectiveness of each.

In order to determine the predictability of success of a recipient mammal, e.g., how well recipient ewes can maintain twin pregnancies, ewes with varying hematocrit and pulse oximetry measurements are used in an embryo transfer experiment where recipient ewes receive 3 embryos and embryos survival rate are determined at 25, 45, and 65 days of gestation as well as number at birth.

### Example 2: Measuring Hematocrit Levels in a Pig Prior to Insemination to Predict Litter Size

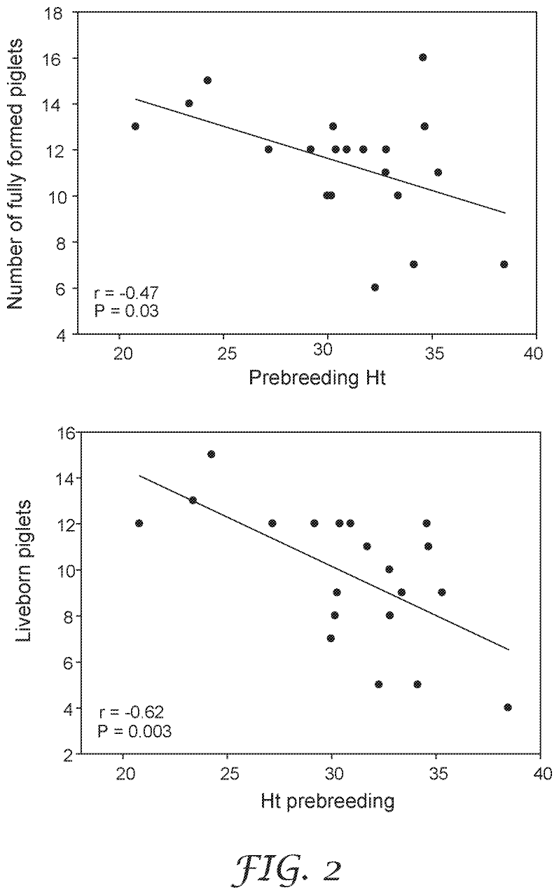

From the data that was collected in the ewe, our objective was to determine if we could predict litter size in the pig during early pregnancy or prior to insemination. Our experiment was to take blood samples (˜10 mL from the jugular vein) on day −2 [with day 0 being day of estrus (i.e. day of breeding)], d 15, 30, 60, and 90 of gestation from the same sows (n=20). We then did correlations with hematocrit values taken on each day with the resulting litter size at birth. We ran statistics on fully formed piglets (live born+still born piglets), and live born piglets. FIG. 2 depicts our findings. Surprisingly, there was no correlation of hematocrit values on days 15, 30, 60 or 90 of gestation, but there was a moderate to strong negative correlation with hematocrit values just prior to breeding with those of resulting litter size. The current hypothesis is that follicular estrogen is altering plasma volume and that this “sets up” the uterus for successful carrying of offspring.

The hypothesis that a short duration of estrogen would increase plamsa volume was tested with ovariectomized ewes (n=12). When the ovaries of a female are removed, the majority of her circulating estrogens is gone. Thirty days after ovariectomy, half of the ewes received an estradiol-17β implant for 24 hours, while the control animals did not. In order to determine if blood volume was altered due to estradiol-17β, a dye (Evan's Blue) was used. There was a tendency (P=0.12) for estradiol-17β treated ewes to have an 8.7% greater plasma volume compared to the control ewes. These data suggest that follicular estrogen at breeding has the potential to expand blood volume, and thus the ability to determine differences of blood volume prior to insemination is viable.

Further studies to determine litter size in swine are to begin with more frequent monitoring of hematocrit and pulse oximetry measurements throughout the estrous cycle and early pregnancy. Determination of litter size is predicted by our measurements.

### Example 3: Measuring Hematocrit Levels in a Dairy Cow Prior to Insemination to Predict Fertility

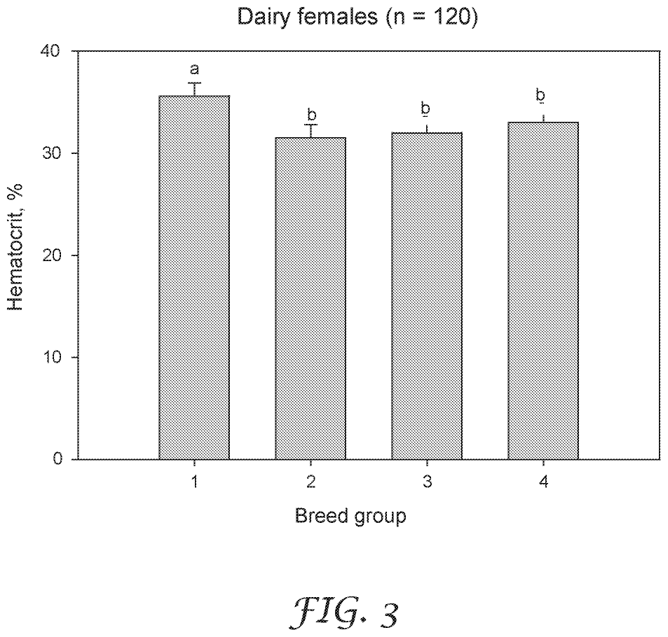

Similar to example 2, we set out to determine if there were differences in hematocrit obtained on the day of breeding (e.g., artificial insemination) and resulting pregnancies in the dairy cow. All cows and heifers at the North Dakota State University (NDSU) dairy farm were artificially inseminated. Females were bred to estrus or are synchronized and timed artificially inseminated. Blood samples (˜10 mL) from the coccygeal vein were obtained on the day of breeding. (A preliminary study determined that jugular ad coccygeal blood were similar in an individual dairy animal). Females were then managed normally and pregnancy diagnosis was determined by the herd's veterinarian. If a female was observed to be in estrus, she was noted and rebred. FIG. 3 depicts data obtained.

These data suggest that on the day of breeding, we could predict if a dairy female could become pregnant based upon her hematocrit values (Breed group 1=females that return to estrus within 22 days; Breed groups 2, 3 and 4 represent cows that do not return to estrus in 22 days, but either return to estrus by day 30 post-insemination [indicative of an early conceptus lost after maternal recognition of pregnancy signal; Group 2] or was determined to be not pregnant by ultrasonography by day 60 [later conceptus loss; Breed group 3], or remained pregnant past 60 days [Breed group 4]). What is not clear by hematocrit values alone at insemination, is whether she will maintain that pregnancy. Further studies are underway to determine how this could be elucidated.

In order to determine that this day was of importance, other days post insemination were evaluated included days 5, 7, 10, 12, 14, 15, 18, 20, 22, 24, 26, 28, 30, (and monthly until term; term=˜280 days).

### Example 4: Measuring Hematocrit Levels in a Ewe Prior to Insemination to Predict Litter Size

Estrus are synchronized in a group of ewes (n=120) and hematocrit levels are determined the day after progesterone-source removal. It is predicted that ewes that have the capabilities to carry twins have reduced hematocrits prior to breeding (breeding will be done by natural service; day of estrus/mating will be monitored and confirmed by lambing date). Moreover, we predict based off FIG. 1, that ewes that do not achieve pregnancy have greater values of hematocrit near the time of estrus.

### Example 5: Measuring Oxygen Saturation Levels in a Mammal Using Oximetry

Another non-invasive means of determining potential plasma volume expansion was to use pulse oximetry. In a pilot study, 4 dairy cows (n=2 nonpregnant and n=2 pregnant) were used. Using pulse oximetry; a sensor was placed on the vulva and it was determined that pregnant cows had a decreased oxygen saturation (77 and 80%) vs non-pregnant cows (95 and 93%). This timing of when pulse oximetry can accurately determine pregnancy needs to be evaluated. Moreover, we predict that pulse oximetry data could be used instead of hematocrit on the day of insemination (in all species) to predict pregnancy success.

We are determining hematocrit (n=25 to 50 ewes) and pulse oximetry data (n>100 ewes) and fetal enumeration/pregnancy success at breeding and at different time points after possible insemination (all ewes will be bred by natural service). Our preliminary evidence suggests that we will determine a difference from unbred ewes during their estrous cycle (FIG. 1 above) compared to pregnant ewes in both hematocrit as well as pulse oximetry measurements. Moreover, we will be able to enumerate the number of fetuses that the ewes are carrying before 3 weeks after insemination.

### Example 6: Measuring Oxygen Saturation Levels in a Mammal Using Oximetry after Insemination to Determine Fetal Viability and Receptivity to Re-Insemination

We predict that if red blood cell volume (i.e. hematocrit) decreases soon after embryonic or fetal loss, this will predict the loss of the pregnancy accurately with either hematocrit or ideally pulse oximetry (ideally because this is non-invasive). During embryonic or fetal loss, there is a period of time that the female will not return to estrus for re-breeding due to her hormonal status. If we can predict embryonic/fetal loss, we can intervene with estrous synchronization drugs to promote a return back to estrus and ovulation in a time that is faster than her body would naturally. This would be particularly useful in the dairy and swine industries as current management methods give producers frequent and easy access to the animals for detection. This does not limit this technology to be used in beef, horse, or sheep operations where producers are more likely to keep animals in facilities that allow for more frequent observations (e.g., dry lot vs pasture settings).

1. A method for predicting the success of pregnancy in a mammal comprising a) obtaining a relevant sample near the time of insemination, b) determining a physiological measurement from the sample and c) comparing the physiological measurement to the same physiological measurement that would be expected for a mammal that will not or has not become pregnant after insemination.

2. The method according to claim 1 wherein the relevant sample is selected from the group consisting of a physiological sample and access to a bodily parameter.

3. The method according to claim 2 wherein the physiological sample is blood.

4. The method according to claim 2 wherein the access to a bodily parameter is access to measure oxygen saturation of blood.

5. The method according to claim 1 wherein the physiological measurement is selected from the group consisting of hematocrit level and oxygen saturation of the blood.

6. The method according to claim 1 wherein the hematocrit level is reduced by at least 15%, the oxygen saturation of the blood is reduced by at least 10%, or a combination thereof.

7. A method for confirming the success of pregnancy in a mammal comprising a) obtaining a relevant sample after the time of insemination, b) determining a physiological measurement from the sample and c) comparing the physiological measurement to the same physiological measurement that would be expected for a mammal that has not become pregnant after insemination

8. A method for predicting litter size in a mammal comprising a) obtaining a relevant sample near the time of insemination, b) determining a physiological measurement from the sample and c) comparing the physiological measurement to those that would be expected for a mammal that would have a litter of known size.

9. A method for identifying qualified candidates for successful embryo transplants from an embryo donor comprising the method according to claim 1.

10. A kit for predicting a successful pregnancy in a mammal comprising a) instructions for collecting relevant samples, b) instructions for obtaining physiological measurements and c) instructions for interpreting the physiological measurements and or bodily parameters to predict a successful pregnancy.

11. A kit for confirming a successful pregnancy in a mammal comprising a) instructions for collecting relevant samples, b) instructions for obtaining physiological measurements and c) instructions for interpreting the physiological measurements and or bodily parameters to confirm a successful pregnancy.

12. A kit for predicting litter size in a mammal comprising a) instructions for collecting relevant samples, b) instructions for obtaining physiological measurements and c) instructions for interpreting the physiological measurements and or bodily parameters to predict a litter size in a mammal.

13. A biochemical device comprising a compact sensor that can analyze a small amount of a relevant sample and determine a physiological measurement in the sample.

14. A biometric device comprising a compact sensor that can determine a physiological measurement in a relevant sample.

15. A biochemical device according to claim 13 wherein the relevant sample comprises a physiological sample.

16. The biometric device according to claim 14 wherein the relevant sample comprises access to a bodily parameter.

17. The method according to claim 13 wherein the compact sensor is in communication with a processing device to generate the physiological measurement and/or analyze a physiological measurement against standards to predict the pregnancy of a mammal and/or size of a litter.
